(19) United States
(12) Patent Application Publication
(10) Pub. No.: US 2005/0074509 A1
(43) Pub. Date: Apr. 7, 2005

(54) Cancer treatment using natural plant products or essential oils extracted from some pistacia species or components

(75) Inventor: Maria Paola Belloni Regazzo, Padova (IT)

Correspondence Address: MODIANO & ASSOCIATI, Via Meravigli, 16, MILANO, 20123, IT

(73) Assignee: DATA MEDICA PADOVA S.p.A.

(21) Appl. No.: 10/676,101

(22) Filed: Oct. 2, 2003

(51) Int. Cl. A61K 35/78

(52) U.S. Cl. 424/769

(57) ABSTRACT

Natural products or pharmaceutical compositions containing plant essential oil, from p. terebinthus, p. lentiscus, p. vera or other pistacia species, or their components, natural or synthetic, or mixture or derivatives thereof, for the prevention and treatment of cancer.

### FIELD OF THE INVENTION

The present invention relates, in general, to therapeutically effective natural products and pharmaceutical compositions containing plant essential oil from pistacia species, or components, for the prevention or the treatment of cancer in mammals, including humans, such as, for example, breast cancer.

### BACKGROUND OF THE INVENTION

It is known that nature represents a large source of therapeutically active drugs (Buffoni 1996). Indeed, the use of plants and other natural products for therapeutic purposes dates back basically to the beginning of humanity (Farnsworth 1985; Cragg. 1997).

Plants, like everything else, can be considered a mixture of different substances which despite a diversified metabolic fate, are maintained in a perfect equilibrium. Various drugs contain active principles with various pharmacologic activities, some of which are fundamental for some specific therapeutic use. Phytotherapy should not thus be considered an alternative cure, but rather an important sector of pharmacotherapy.

In fact, the phytotherapeutical remedies are often associate to the drugs of synthesis which, in various cases, are only to completion of therapies “natural”.

The natural products represented for time an excellent source of medicinal for the care of the cancer. The anti-tumorals of natural origin which are used successfully in the clinical practice are therefore several and some of these also very well known such as, for example, taxol, isolated from Taxus brevifolia; vincristine and the vinblastine, isolated from Vinca rosea; etoposide and teniposide, semisynthetic-derivatives of podophyllotoxin, isolated from Podophyllum peltatum. Natural products, moreover, given their structural variety, continue to attract interest in the antitumoral field (Farnsworth, 1990; Cragg 1999).

Among plant natural products having potential pharmacological activity, essential oils, pure mixtures of organic substances, play a central role. Even in the same species the composition of an essential oil is very variable due to the plant high sensitivity to different climatic conditions. Essential oils are generally obtained by compression or hydro-distillation. The distillation in steam current is the most widely used extraction method. Given the very complex composition of essential oils and the many quantitative changes occurring during the vegetative cycle of the plant, their characterization is quite difficult.

The Pistacia genus (Anacardacee) includes several species and is constituted by bushes or small trees, shrubs with resinous cortex.

The species found in the Mediterranean area are:
P. vera
P. terebinthus
P. lentiscus

The oil obtained by the Pistacia Vera seeds is scarcely used and is present only in a limited number of pharmaceutical preparations, while, essential oil extracted from the resin of Pistacia Terebinthus has been shown to exert a significant anti-inflammatory activity in an experimental model of auricular inflammation in the rat (Giner-Larza 2000). Certainly much more used is instead the drug called rubber or mastic of lentiscus extracted by the Pistacia Lentiscus. This is used in pills as expectorant, while its tonic and astringent action is exploited for the care of children's diarrheas. Mastic is also chewed for its light antiseptic oral activity and sometimes it is associated to camphor, sandracca and peruvian balm to eliminate bad breath. The finding of the lentiscus resin in mummies dating back to the seventh century A. C. shows that the Egyptians used this substance to embalm the dead exploiting its antiseptic activity (Colombini 2000).

It is also known that:
the water extract of Pistacia Lentiscus, rich of potassium, sodium and magnesium, induces in the rat a hypotensive activity (Sanz 1987, 1988), probably due to the presence of n-butanolic and ethyl in the extract;
the essential oil obtained from the hydrodistillation of the resin of the resin of lentiscus exerts an antibacteric activity in vitro more marked toward the gram +, respect to gram − bacteria. Identifying, in the essential oil, a “natural” antibacteric action is of interest also because it could replace preserving substances, often suspected of toxicity, cancerogenity and theratogenity (Magiatis 1999);
among the various extracts obtained by freeze-dried P. lentiscus leaves, the decoction is the only one to have in vitro, a good antibacteric activity in cultures of Staphylococcus aureus, Sarcina lutea and Escherichia coli and have a modest antimycotic activity, proven on cellular Torulapsis glabrata and Candida Parapsilosiscoltures;
the resin of the lentiscus, even when used at low dosages, acts quickly against peptic ulcer, thanks to its effectiveness against Helicobacter Pylori (Huwez 1998, Marone 2001).
the cortex and leaves from Pistacia lentiscus are used against diarrhea and gonorrhea.

Essential oils from the pistacia genere are rich in monoterpenes, which in fact represent the major components. Monoterpenes are non-nutritive dietary components also found in the essentials oils of many edible plants such as citrus, cherry, spearmint, dill, caraway, and others. Their natural functions may be as chemoattractants or chemorepellents, as they are largely responsible for the plant's pleasant fragrance. These simple 10 carbon isoprenoids are derived from the mevalonate pathway in plants but are not produced in mammals. For example, in spearmint and other plants, d-limonene is formed by the cyclization of geranylpyrophosphate by the enzyme limonene synthase (Croteau 1987). Limonene then serves as a precursor for other plant monocyclic monoterpenes such as carvone, carveol, and perillyl alcohol (Elson 1994).

The antitumor effects of dietary monoterpenes are attained with little or no host toxicity (Elson 1994, Crowell 1994 a, b, Evans 1995). A number of dietary monoterpenes have antitumor activity, exhibiting not only the ability to prevent the formation or progression of cancer, but to regress existing malignant tumors. Limonene and perillyl alcohol have well established chemopreventive activity against many cancer types. Indeed, d-limonene has a broad range of antitumor activities (Elson 1994, Crowell 1994). Dietary limonene reduces the incidence of spontaneous lymphomas in p53^{-/-} mice (Hursting 1995). Limonene, besides, has chemopreventive activity against spontaneous and chemically-induced rodent mammary, skin, liver, lung, and fore-stomach cancers, as well as ras oncogene-induced rat mammary cancer (Gould 1994). Furthermore, when administered either in pure form or as orange peel oil (95% d-limonene), limonene inhibits the development of chemically induced rodent mammary (Elegbede 1984, Elson 1988, Maltzman 1989, Wattenberg 1983), skin (Elegbede 1986 a), liver (Dietrich 1991), lung and forestomach (Wattenberg 1989, 1991) cancers (reviewed in Crowell and Gould 1994, Elson and Yu 1994, Elson 1995). In rat mammary carcinogenesis models, the chemopreventive effects of limonene are evident during the initiation phase of 7-12-dimethylbenz[a]anthracene (DMBA)²—induced cancer (Elson 1988) and during the promotion phase of both DMBA—and nitrosomethylurea (NMU)—induced cancers (Elson 1988, Maltzman 1989). Kawamori et al. (1996) reported that the development of azoxymethane-induced aberrant crypt foci in the colon of rats was significantly reduced when they were given 0.5% limonene in the drinking water. A Phase I clinical trial testing limonene's cancer chemotherapeutic activity is in progress (McNamee 1993).

Caraway seed oil, and its principal monoterpene, carvone, prevent chemically induced lung and forestomach carcinoma development when administered before the carcinogen (Wattenberg 1989). In addition, carveol (Crowell 1992) and menthol (Russin 1989) have chemopreventive activity against DMBA-induced rat mammary cancer when fed as 1% of the diet only during the initiation phase. Geraniol, an acyclic dietary monoterpene, has in vivo antitumor activity against murine leukemia, hepatoma and melanoma cells (Shoff 1991, Yu 1995) when administered before and after tumor cell transplantation.

In addition, many animal studies have shown perillyl alcohol to be a very powerful chemotherapeutics agent against several cancer types including pancreatic, breast, and liver cancer (Crowell 1999) and to have promotion phase chemopreventive activity against chemically induced liver cancer in rats (Mills 1995) and to be very effective at preventing tumor recurrences or secondary tumors in animals treated in a chemotherapy regimen (Haag 1994). Perillyl alcohol has chemotherapeutic activity against pancreatic cancer at doses that cause little toxicity to the host (Stark 1995). Perillyl alcohol reduced the growth of transplanted hamster pancreatic tumors to less than half that of controls. Moreover, a significant portion of perillyl alcohol-treated pancreatic tumors completely regressed, whereas none of the control tumors regressed (Stark 1995). Perillyl alcohol chemotherapy also reduces the growth rate of transplanted prostatic carcinomas in nude mice (Jeffers 1995). Thus, monoterpenes have chemotherapeutic activity against a number of solid types, including pancreatic cancer, one of the most refractory of all human cancers to available cancer therapies. The efficacy of perillyl alcohol chemotherapy against human cancer will be tested in forthcoming Phase I clinical trials (Phillips 1995)

Both limonene (Elegbede 1986 b, Haag 1992) and perillyl alcohol (Haag 1994) have chemotherapeutic activity against rat mammary tumors, causing the complete regression of >80% of established DMBA- or NMU-induced rat mammary tumors with limonene and the aromatase inhibitor 4-hydroxyandrostenedione was more effective than either drug alone.

Several mechanisms of action may account for the chemotherapeutic activities of monoterpenes. The blocking chemopreventive effects of limonene and other monoterpenes during the initiation phase of mammary carcinogenesis are likely due to the induction of Phase II carcinogen-metabolizing enzymes, resulting in carcinogen detoxificatoin. The post-initiation phase, tumor suppressive chemopreventive activity of monoterpenes may be due in part to the inhibition of isoprenylation of cell-growth associated small G proteins such as p21 ras by limonene, perillyl alcohol, and their metabolites (Crowell 1991, 1994). This inhibition occurs at the level of the prenyl-protein transferases. In addition, perillyl alcohol affects the mevalonate pathway by inhibiting ubiquinone biosynthesis as well as the conversion of lathosterol to cholesterol (Ren 1994). Chemotherapy of chemically-induced mammary tumors with monoterpenes results in tumor redifferentiation (Haag 1992). In limonene-treated mammary tumors, expression of the mannose-6-phosphate-insulin-like growth factor II receptor and transforming growth factor β1 are increased in the regressing, differentiating tumors, but not in the small number of tumors which are unresponsive to limonene (Jirtle 1993). In addition, the antitumor effects of dietary monoterpenes are attained with little or no host toxicity (Elson 1994, Crowell 1994, a,b, Evans 1995). In summary, a variety of dietary monoterpenes have been shown to be effective in the chemoprevention and chemotherapy of cancer. Now, monoterpenes research is progressing into human clinical trials for chemotherapeutic activity. Monoterpenes also possess many characteristics of ideal chemopreventive agents, namely, efficacious antitumor activity, commercial availability, low cost, oral bioavailability, low toxicity and novel mechanisms of action different from those of conventional cancer chemotherapeutic drugs, making it feasible to begin considering them for human cancer chemoprevention testing (Crowell 1996).

Several studies were led to the purpose to identify the chemical composition of the oil obtained by the leaves of P. lentiscus. Concluding that at second of the geographic origin area the various oils are characterized by a monoterpene unusual, the myrcene is present in particular by 19-25% in the oil coming from Spain and the Sicily (Calabro 1974, Boelens 1991) is possible from the analysis of the studies taken back in literature; the α-pinene is present for 16% in those French (Buil 1975); the terpen-4-ol is present by 22% in the one coming from the Sardinia (Castola 2000) and δ-3-carene the Egyptian oil characterizes (65%) (De Pooter 1991). Present members in less amount are a few sesquiterpeni, what: D-germacrene (9%) (Boelens 1991), the β-caryophyllene (3.5-9%) (Buil 1975, Boelens 1991), δ-cadinene and α-cadinolo (6% of everybody) (Buil 1975), the β-bisabolene, β-bourbonene and caryophyllene oxide (about 3-4% of everybody) (De Pooter 1991). The concentrations of the monoterpenes, besides, significantly change if the oil is obtained by the fruit. In particular, comparing two oils, the one coming from the Spain (Boelens 1991) and the one from the Australia (Wyllie 1990), it obtains, what component majors, respectively myrcene (72 and 39%), α-pinene (10 and 28%) and the limonene (87 and 11%).

The oils obtained for hydrodistillation by the mastic coming from Spain and Greece are instead characterized by a high α-pinene content (65-86%) and a low myrcene (3-25%) content (Scurbis 1975, Papageorgiou 1981; Katsiotis 1984; (Boelens 1991).

Of particular interest is the work of Migiatis and coll. (1999) who, using gas-cromatography and mass spectroscopy, identified 69 members of essential treols of P. lentiscus, var. chia, respectively obtained from the leaves, the twigs and the mastic.

### SUMMARY OF THE INVENTION

The present invention deals with the use of pistacia natural products or essential oils and/or components, natural or synthetic, or mixtures or derivatives, and possibly other related natural products thereof for cancer prevention and treatment. In particular the present invention relates to the use of the above mentioned, by either oral or parenteral administration, also as adjuvant in combination with other cures, in preventive and therapeutic regimens directed towards the inhibition of cell growth or the killing of tumoral cells in humans and other animal species.

Additional objects and attendant advantages of the present invention will be set forth, in part, in the description that follows, or may be learned from practicing or using the present invention. The objects and advantages may be realized and attained by means of the features and combinations particularly recited in the appended claims. It is to be understood that the foregoing general description and the following detailed description provides the experimental basis for the invention, are exemplary and explanatory only and are not to be viewed as being restrictive of the invention, as claimed.

### DESCRIPTION OF THE DRAWINGS

FIG. 1 shows the cytotoxic effect of Lentiscus oil from Portugal.

FIGS. 2a, 2b and 2c show the cytotoxic effect of single oil components.

FIG. 3 shows the cytotoxic effect of DM1, DM1P and DM1 S on MCF-7 cells.

FIG. 4 shows the cytotoxic effect of DM2A1, DM1Z and DM3Z on 2008 cells.

FIG. 5 shows results of cytofluotrimetric analysis.

FIG. 6 shows an evaluation of the nitrite concentration.

### DETAILED DESCRIPTION OF THE INVENTION

The present invention regards a method for treating or preventing cancer in a mammal, including a human, comprising administrating an effective amount of a product obtained from a plant of Pistacia genus.

“Products obtained from a plant of the Pistacia genus” as used in the present specification and claims, means any part of a plant of Pistacia genus, as leaves, twigs, seeds, routs, galls, fruits, any natural product of a plant of pistacia genus, as resins, products obtained from a plant of the Pistacia genus by any technique, for example but not limited to extraction, grinding, chemical, physical or chemical-physical treatments.

In one embodiment of the present invention, the products are products containing essential oils from plants of Pistacia genus, essential oils of plants of Pistacia genus as such or components thereof.

“Plant of Pistacia genus” as used in the present specification and claims means any plant of the Pistacia genus, of any geographical origin and of any species. In one embodiment of the present invention, the plant of Pistacia genus is of European origin. In some embodiments of the present inventiion, the plant is of one of the species p. Terebinthus, p. Lentiscus and p. Vera.

The present invention particularly deals with the use of pistacia natural products or essential oils and/or components, natural or synthetic, or mixtures or derivatives, and possibly other related natural products thereof for cancer prevention and treatment. In particular the present invention relates to the use of the above mentioned, by either oral or parenteral administration, also as adjuvant in combination with other cures, in preventive and therapeutic regimens directed towards the inhibition of cell growth or the killing of tumoral cells in humans and other animal species.

### BRIEF DESCRIPTION OF EXPERIMENTAL DATA

The features and advantages of the present invention will become more clearly appreciated from the following description of experimental data indicating the in vitro anti-tumoral activity of essential oils extracted from various species of Pistacia.

Methods:

Plant Collection:

Aerial parts (twigs, branches, leaves, fruits, seeds, flowers and galls) of the plants were collected in different seasons and at various times of the day in three different Italian regions and in particular:

Pistacia terebinthus: Veneto

Pistacia lentiscus: Tuscany

Pistacia vera: Sicily

Plant parts were collected and then rinsed, dried and frozen at −80° C. within three hous from collection. The material was then hydro-distilled within few months. The vegetable material exposed to hydro-distillation consisted in leaves, flowers, fruits, branches and galls of P. terebinthus P. lentiscus and P. Vera. Samples were washed and dried carefully and preserved to low temperature (−21°) to keep unchanged their phytochemical composition until the moment of the distillation. All the samples, before being exposed to distillation, were minced to obtain the maximum extraction yield and make the process of diffusion of the essence easier.

Essential Oil Extraction

The equipment used for the extraction of the essential oils consisted in a container in Inox steel (10 l), in which 1750 ml of distilled water are added, separated from the minced drug by a steel grid, to avoid direct contact of the drug with the extraction water. The sample is compacted by a further grill to avoid handling of the drug during the extraction proceeding and at the same time let the water steam freely flow down. To this point the steel container closed. The boiler is equipped with a thermometer, to be able to check at every moment of the distillation the temperature in boiler, and is also connected to a distillation column in steel, taking a coolant to the superior extremity, always in steel, with cooling running water. Water in the range container to the ebullition develops steam which going beyond the grill, laps the drug and extracts the essences contained, gone beyond the grill, the steam is directed along the distillation column and condensed in the coolant; the water mixture-essences to this point is collected in a graded cylinder containing some ethilic ether to dissolve the essential oil extracted by the steam, which for their liophylic nature, present a greater affinity for the solvent. The organic phase is then treated with natrium sulphate anhidrous, filtered and evaporated. The distillation was usually led for up to 4 hours with constant heating; the mean initial weight of the sample was: leaves (450 g), branches (250 g), fruits (100 g), flowers (270 g). The yield for the various parts of the plant, express as percent of initial wet weight was: leaves (0,05%), branches (0,06%), fruits (0,11%), flowers (0,08%), galls (0,40%).

Determination of Oil Chemical Composition

The chemical composition of the essential oils obtained by P. lentiscus, P.terebinthus and P. Vera was determined by means of analysis gascromatographic coupled to a detector mass spectrophotometer (GC/MS), using an operating system Hewlett-Packard 6890-5973 in endowed modality El (electronic ionization with potential 70 eV), equipped of capillary columne HP-5 MS (30 m×0.25 mm), with thickness of the equal film to 0.25 m, stationary phase of polidimetil silossano al 95%. It was operated applying one program of temperature starting from 60° C. for the first three minutes raising up to 280° with a speed of 3° C./min. for 5 minutes; the Injector was kept to 200° C.

Biological Assays

Samples preparation: the stock solution of essential oils (9%) were prepared in DMSO (1%) and in culture medium (90%). All the procedures were carried out under sterile conditions. Before each experiment the stock solutions were diluted with growth medium and used immediately.

Cell Lines: the human breast adenocarcinoma cells lines MCF-7, supplied by the Istituto Zooprofilattico Sperimentale della Lombardia e dell'Emilia (Brescia, Italy), were cultured in MEM with Eagle's salts, plus 10% heat-inactivated fetal calf serum, 1% antibiotics and sodium piruvate (all products of Biochrom KG Seromed), 1% 200 mM glutamine (Merck).

The human colon adenocarcinoma cell line LoVo, kindly supplied by Dr. G. Toffoli, Oncologic Reference Centre, Aviano, Italy. Tle cell line was cultured in Ham's F12 with the Addition of 10% heat inactivated Foetal Calf Serum, 1% glutamine 200 mM (Merk), 1% natrium piruvate (Seromed Biochrom KG, Berlin).

The human ovarian adenocarcinoma cell line 2008, kindly supplied by Prof. G. Marverti (Department of Biomedical Sciences, University of Modena), were maintained in RPMI 1640 supplemented with 10% heat-inactivated FCS, 1% antibiotics (all products of Biochrom KG Seromed), and 2 mM 1-glutamine (Merck).

Cytotoxicity: The cells (1×10⁵ cells/ml) were seeded in 96-well tissue plates (Falcon) and treated 24 h later with each essential oil at different concentrations. After 3 h exposure, medium was discarded, the plates were washed with sterile PBS and then added with growth medium.

The cytotoxic effect was evaluated was by tetrazolium salts reduction assay (MTT) after 21 h of incubation. An amount of 20 μL of MTT solution (5 mg/mL in PBS) was added to each well, and plates were incubated for 4 h at 37° C. DMSO (150 μL) was added to all wells and mixed thoroughly to dissolve the dark-blue crystals. The absorbance was measured on a microculture plate reader (Titertek Multiscan) using a test wavelength of 570 nm and a reference wavelength of 630 nm.

Nitrite assay: The nitrite concentration in the culture medium was measured as an indicator of NO production using Griess reaction. One hundred microliters of each supernatant was mixed with the same volume of Griess A reagent (1% sulphanilamide in 5% phosphoric acid) and after 10 minutes 100 ml of Griess B reagent (0.1% naphthylethylenediamine dihydrochloride in water) was added. After 15 minutes the absorbance of mixture was determined at 543 nm.

Cytofluorimetry: cells are collected and rinsed twice with cold PBS and then resuspended in 1× binding buffer at a concentration of 1×106 cells/ml. Transfer 100 μl of the solution (1×105 cells) to a 5 ml culture tube. 5 μl of Annexin V-FITC and 5 μl of PI are added. Cells are gently vortexed and incubated for 15 min at RT (25° C.) in the dark. 400 μl of 1× binding buffer to each tube are then added and samples analyzed by flow cytometry within one hour. Annexin V is a 35-36 kDa Ca2+ dependent phospholipid-binding protein that has a high affinity for PS, and binds to cells with exposed PS. Annexin V may be conjugated to fluorochromes such as Propidium Iodide (PI). This format retains its high affinity for PS and thus serves as a sensitive probe for flow cytometric analysis of cells that are undergoing apoptosis. Since externalization of PS occurs in the earlier stages of apoptosis, Annexin V-FITC staining can identify apoptosis at an earlier stage than assays based on nuclear changes such as DNA fragmentation. Annexin V-FITC staining precedes the loss of membrane integrity which accompanies the latest stages of cell death resulting from either apoptotic or necrotic processes. Therefore, staining with Annexin V-FITC is typically used in conjunction with a vital dye such as Propidium Iodide to allow the investigator to identify early apoptotic cells (Annexin V-FITC positive, PI negative). For example, cells that are viable are Annexin V-FITC and PI negative; cells that are in early apoptosis are Annexin V-FITC positive and PI negative; and cells that are in late apoptosis or already dead are are both Annexin V-FITC and PI positive. This assay does not distinguish, per se, between cells that have already undergone apoptotic death and those that have died as a result of a necrotic pathway because in either case, the dead cells will stain with both Annexin-FITC and PI. However, when apoptosis is measured over time, cells can be often tracked from Annexin V-FITC and PI negative (viable, or no measurable apoptosis), to Annexin V-FITC positive and PI negative (early apoptosis, membrane integrity is present) and finally to Annexin V-FITC and PI positive (end stage apoptosis and death). The movement of cells through these three stages suggests apoptosis. In contrast, a single observation indicating that cells are both Annexin V-FITC and PI positive, in of itself, reveals less information about the process by which the cells underwent their demise.

Reagents

1. Annexin V-FITC
2. Propidium Iodide.
3. 10× Annexin V Binding Buffer.
   Staining
1. Wash cells twice with cold PBS and then resuspend cells in 1× binding buffer at a concentration of 1×106 cells/ml.
2. Transfer 100 μl of the solution (1×105 cells) to a 5 ml culture tube.
3. Add 5 μl of Annexin V-FITC and 5 μl of PI.
4. Gently vortex the cells and incubate for 15 min at RT (25° C.) in the dark.
5. Add 400 μl of 1× binding buffer to each tube. Analyze by flow cytometry within one hour.

The results are shown in Table 1a and Table 1b.

TABLE 1a
Oil Composition

| | P. LENTISCUS | | | | P. VERA |
|---|---|---|---|---|---|
| | Twigs | Leaves | | | Seeds |
| COMPOUND | PORTUGUESE % | DMIC % | DM2A1 % | DM3Z % | DM1S % |
| α-Phellandrene | — | 1.51 | <1 | <1 | — |
| α-Pinene | 12.62 | 11.45 | 12.97 | 12.14 | 0.99 |
| Camphene | 2.12 | 1.46 | 2.49 | <1 | — |
| Sabinene | <1 | 4.31 | 4.56 | 3.48 | — |
| β-Pinene | 3.82 | 3.07 | 4.38 | 1.76 | — |
| β-Myrcene | 8.40 | <1 | <1 | 7.81 | — |
| 1 Phellandrene | — | 4.64 | <1 | 4.59 | — |
| α-Terpinene | — | 4.66 | 3.60 | 3.99 | — |
| p-Cymene | 7.79 | <1 | <1 | <1 | — |
| β-Phellandrene | — | 6.48 | 4.93 | 6.29 | — |
| Limonene | 18.00 | — | — | — | 1.54 |
| Cis Ocimene | — | <1 | <1 | 1.16 | — |
| Trans Beta Ocimene | — | <1 | <1 | <1 | — |
| γ-Terpinene | — | 7.42 | 5.90 | 6.07 | — |
| α-Terpinolene | — | 2.48 | 2.08 | 2.41 | — |
| Endoborneol | 6.90 | <1 | <1 | — | — |
| 1-4-Terpineol | 3.08 | 14.89 | 12.26 | 11.17 | 2.13 |
| α-Terpineol | <1 | 4.92 | 5.36 | 4.83 | 2.56 |
| Bornyl Acetate | 28.19 | 2.12 | 1.77 | — | — |
| α-Copaene | — | <1 | 1.03 | 1.08 | — |
| β-Caryophyllene | 1.63 | 2.79 | 3.21 | 2.76 | <1 |
| α-Humulene | — | 1.02 | <1 | 1.03 | — |
| α-Amorphene | <1 | <1 | 1.51 | 1.17 | — |
| Germacrene D | — | 4.71 | 6.82 | 4.46 | <1 |
| α-Muurolene | — | <1 | 1.21 | <1 | — |
| δ-Cadinene | — | 2.82 | 4.80 | 3.85 | <1 |
| Butylated Hydroxy Toluene | — | <1 | <1 | <1 | 15.74 |
| γ-Eudesmol | — | 1.21 | <1 | <1 | — |
| α-Cadinol | <1 | 2.50 | 3.29 | 3.51 | — |
| β-Caryophyllene Oxide | 2.53 | — | — | — | — |
| T Cadinol | <1 | 3.16 | 3.90 | 3.86 | — |
| 4 Chloro 2 Phenylaniline | — | — | — | — | 32.88 |
| Benzene 1 Methoxy 2,3,5 Trimet | — | — | — | — | 25.63 |
| 4,12 BisHydroxymethyl | — | — | — | — | 4.29 |

TABLE 1b
Oil Composition

| | P. TEREBINTHUS | | | | | |
|---|---|---|---|---|---|---|
| | Galls | | Leaves | Twings | Fruits | |
| COMPOUND | DM1P % | DMG1 % | DM1Z % | DMF3 % | DMF2 % | DMK % |
| α-Phellandrene | — | — | — | — | — | — |
| α-Pinene | 42.16 | 54.19 | 32.50 | 40.88 | 8.69 | 26.84 |
| Camphene | <1 | <1 | <1 | 1.01 | <1 | 1.31 |
| Sabinene | <1 | <1 | — | — | <1 | <1 |
| β-Pinene | 1.81 | 3.68 | 1 | 6.14 | 1.39 | 7.06 |
| β-Myrcene | 1.42 | 1.05 | 1.54 | 1.46 | 1.11 | 1.42 |
| 1 Phellandrene | 6.61 | <1 | <1 | <1 | 7.66 | 3.51 |
| α-Terpinene | — | — | — | <1 | <1 | <1 |
| p-Cymene | <1 | — | — | — | — | <1 |
| β-Phellandrene | — | — | — | 6.14 | — | — |
| Limonene | 31.07 | 13.97 | 23.75 | — | 32.85 | 12.48 |
| Cis Ocimene | <1 | 1.89 | 26.50 | 10.95 | 17.99 | 7.84 |
| Trans Beta Ocimene | — | <1 | 7.48 | 3.89 | 5.01 | 1.89 |
| γ-Terpinene | — | <1 | <1 | <1 | <1 | <1 |
| α-Terpinolene | <1 | <1 | <1 | 16.96 | 1.27 | <1 |
| Endoborneol | — | — | — | — | — | — |
| 1-4-Terpineol | <1 | <1 | — | <1 | <1 | 1.04 |
| α-Terpineol | 1.36 | <1 | 1.39 | 5.24 | <1 | 3.24 |
| Bornyl Acetate | <1 | — | — | <1 | <1 | <1 |
| α-Copaene | <1 | <1 | <1 | — | <1 | 5.15 |
| β-Caryophyllene | <1 | 14.33 | 2.01 | 2.66 | 1.64 | 5.38 |
| α-Humulene | <1 | 2.15 | <1 | — | <1 | 1.66 |
| α-Amorphene | <1 | — | — | — | <1 | <1 |
| Germacrene D | 7.08 | 2.91 | <1 | — | 9.42 | 2.37 |
| α-Muurolene | — | — | — | — | — | <1 |
| δ-Cadinene | <1 | <1 | <1 | — | 2.33 | 7.36 |
| Butylated Hydroxy Toluene | <1 | <1 | <1 | <1 | <1 | <1 |
| γ-Eudesmol | <1 | — | — | — | — | — |
| α-Cadinol | <1 | — | — | — | — | — |
| β-Caryophyllene Oxide | — | <1 | — | — | — | <1 |
| T Cadinol | <1 | — | — | — | <1 | <1 |
| 4 Chloro 2 Phenylaniline | — | — | — | — | — | — |
| Benzene 1 Methoxy 2,3,5 Trimet | — | — | — | — | — | — |
| 4,12 BisHydroxymethyl | — | — | — | — | — | — |

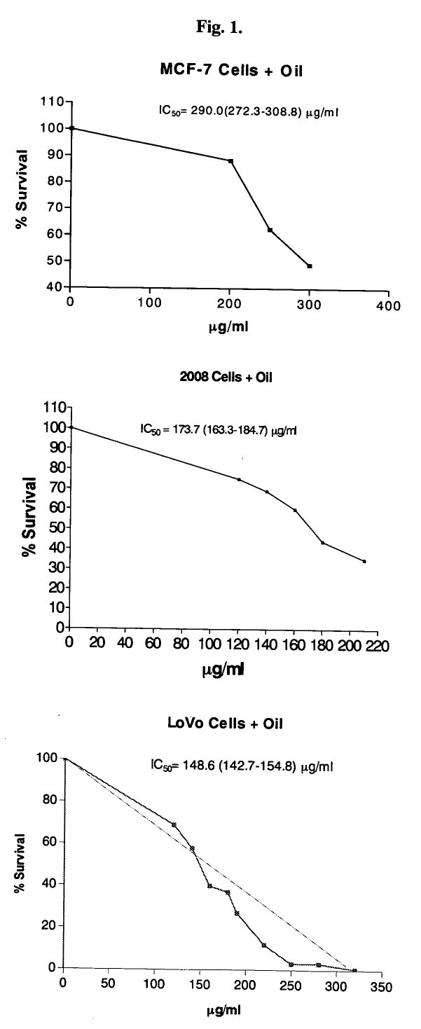

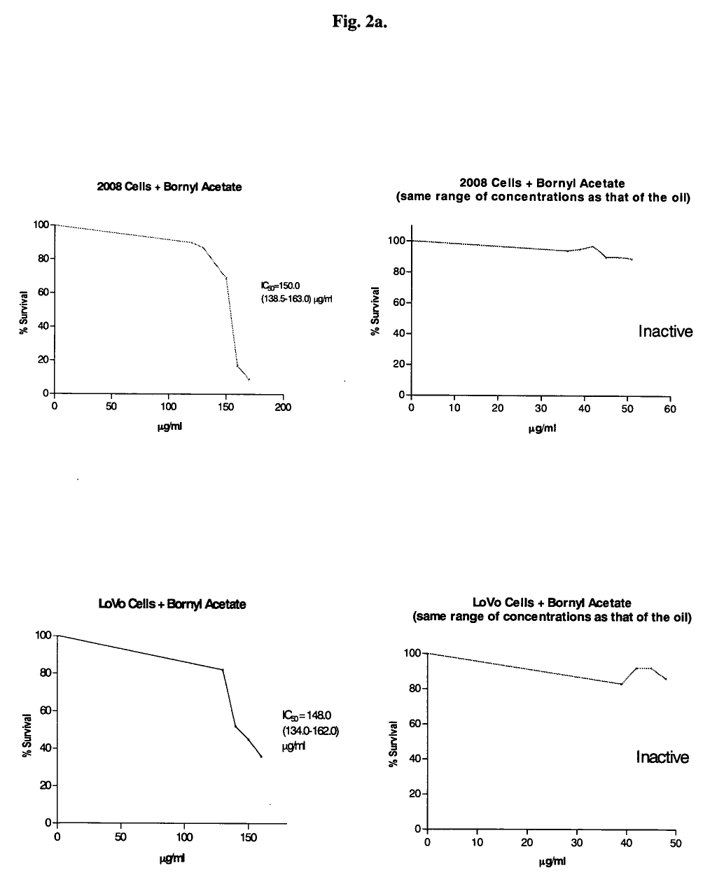

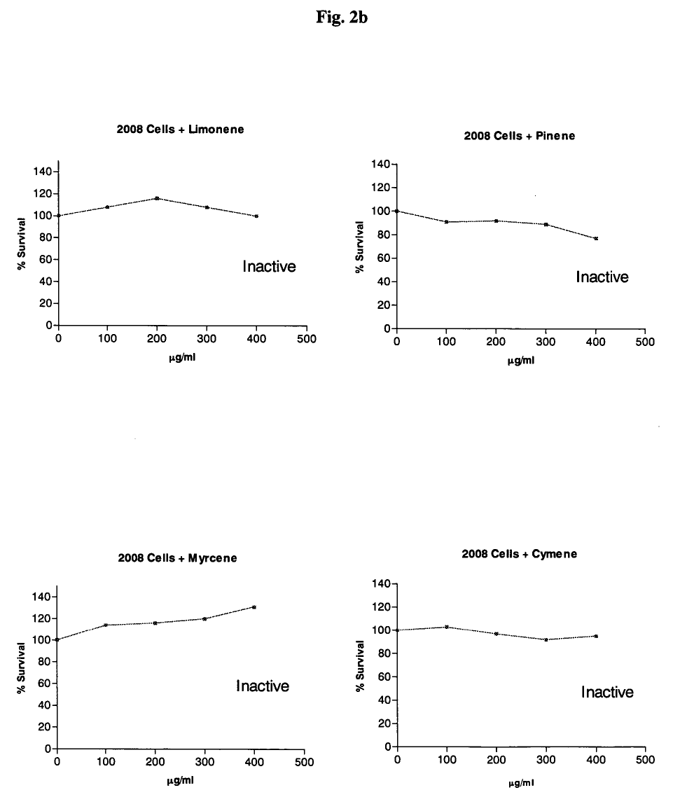

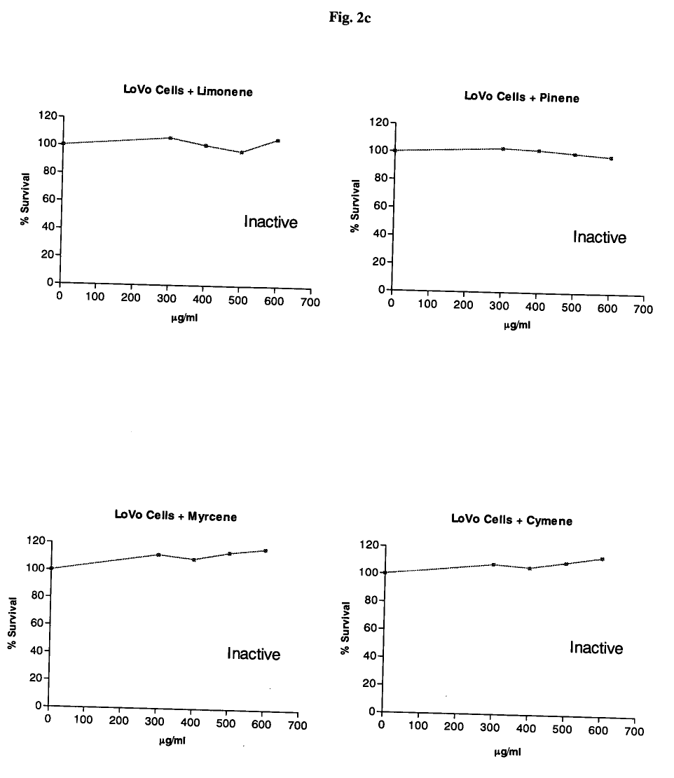

Following are some examples of results obtained evaluating the cytotoxicity of several essential oils against the selected tumor cell lines (MCF-7, LoVo and 2008) and showed that the oil from twigs of Portuguese lentiscus (see Table 1a and 1b for composition) was active in inducing a cytotoxic effect (FIG. 1) with IC₅₀ of 290 (272.3-308.8) μg/ml on MCF-7, 173.7 (163.3-184.7) μg/ml on 2008 and 148.6 (142.7-154.8) μg/ml on LoVo cells. The cytotoxic effect was also assayed using some of the single components of the oil and the only active tested component, in our experimental conditions, was Bomyl Acetate (FIG. 2), but when this compound was utilized at the equivalent concentration of the oil it resulted inactive (FIG. 2). Among the single components tested, limonene, which has been reported in literature as having antitumoral effect, did not prove active in our experimental conditions.

Other results obtained using oils from Italian pistacia showed that ten oils were able to reduce cell growth. In particular, DMF3 (oil extracted from one sample of P. terebinthus twigs) resulted active in inducing cytotoxicity in both MCF-7 and 2008 cell lines with IC₅₀ of 474.7 (505.2-617.8) μg/ml, respectively.

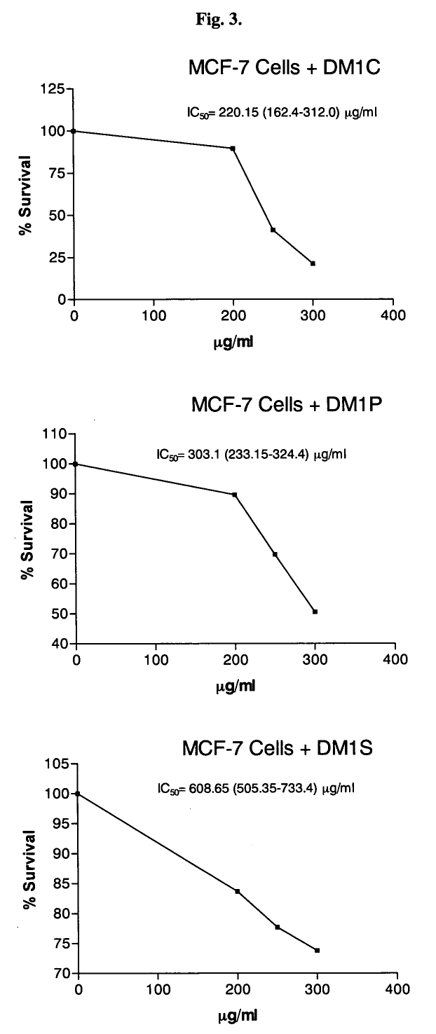

DM1C (oil extracted from one sample of P. lentiscus leaves), DM1P, DMG1 (oils extracted from two samples of P. terebinthus galls) and DM1S (oil extracted from one sample of P. vera seeds) showed a significant activity on human breast adenocarcinoma cells MCF-7 (FIG. 3). The results expressed as IC₅₀, are: 220.1 (162.4-312.0) μg/ml, 303.1 (233.1-324.4) μg/ml, 503.3 (470.9-537.9) μg/ml and 608.6 (505.3-733.4) μg/ml, respectively, while IC₅₀ calculated for portuguese oil, taken as reference compound, was 290.0 (272.3-308.8) μg/ml. Thus, two oils, DM1C and DM1P, showed the same cytotoxic effect respect to the portuguese oil, while DMG1 and DM1 S had a milder toxicity.

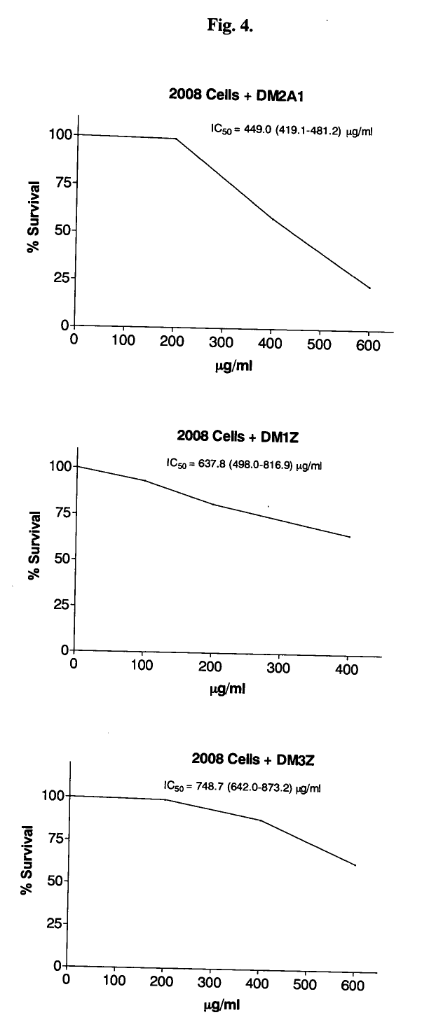

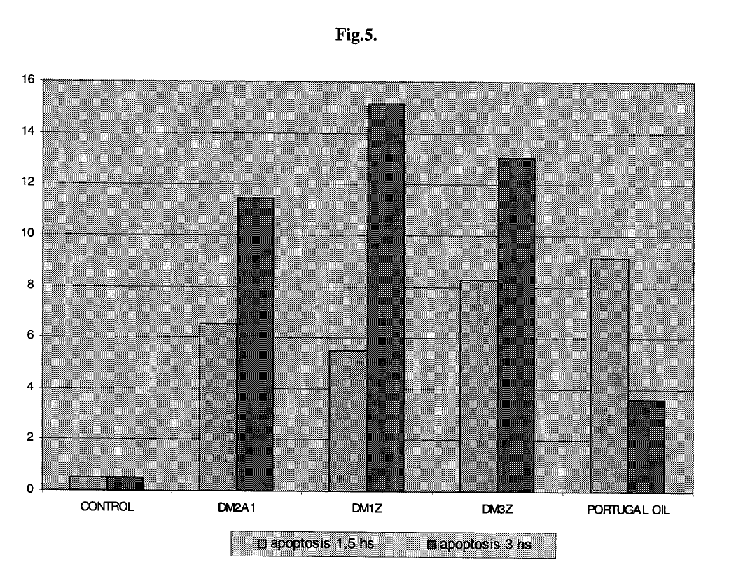

DMF2 and DMK (oils extracted from two samples of P. terebinthus fruits) were active on human ovarian adenocarcinoma cells 2008 with IC₅₀ of 429.5 (382.2-482.6) μg/ml and 520.9 (487.6-556.4) μg/ml, respectively. DM2A1 (oil extracted from one sample of P. lentiscus leaves), DM1Z (oil extracted from one sample of P. terebinthus leaves) and DM3Z (oil extracted from one sample of P. lentiscus leaves) were active on human ovarian adenocarcinoma cells 2008 (FIG. 4) with IC₅₀ of 449.0 (419.1-481.2) μg/ml, 637.8 (498.0-816.9) μg/ml and 748.7 (642.0-873.2) μg/ml, respectively. The activities of these last three oils as well as that of the oil from Portugal were also tested with two cytofluorimetric assays to analyse the nature of cell death (Annexin V plus Propidium Iodide; NO production), which resulted mostly in apoptotic death for all oils. (FIG. 5).

The cytofluometric tests indicated a cytotoxic effect with the same oils (FIG. 5).

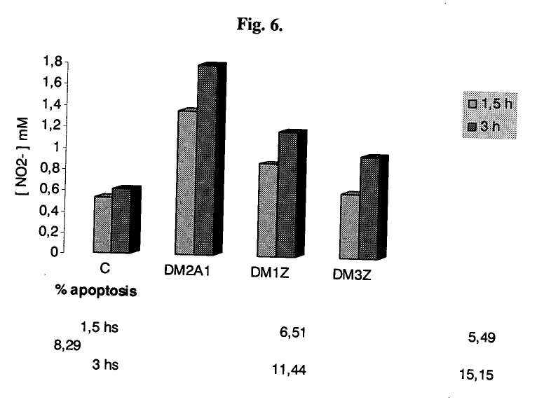

The results obtained evaluating the nitrite concentration in the culture medium of 2008 cells treated for 1.5 and 3 h with DM2A1, DM1Z and DM3Z indicated the activation of apoptotic mechanisms. It should be pointed out that these results were in accordance also to the cytotoxicity studies, indeed increasing the NO detected in medium, increasing the cytotoxic effect: DM2A1 was the most cytotoxic oil on 2008 cells and was able to induce the most production of NO (FIG. 6).

### REFERENCES

Boelens M. H., Jemenez R. Chemical composition of the essential oil from the gum and various parts of Pistacia lentiscus L. (Mastic Gum Tree). Flav. Fragr.J, 6:271-275,1991.
Buffoni F. La natura come fonte inesauribile di farmaci. Acta Phytotherapeutica., 2,3-6,1996.
Buil P. et al. Contribution a la connaissance de la composition chimique de l'essence de lentisque de Provence. Riv. Ital. EPPOS Cosmet. Aerosol., 56:245-252,1975.
Calabro G., Curro P. Costituenti degli oli essenziali Nota IV. Essenza di lentisco. Essence Deriv.Agrum., 44:82-92,1974.
Castola V. et al. Intraspecific chemical variabilita of the essential oil of Pistacia lentiscus L. from Corsica. Biochem. System. Ecoli., 28:79-88,2000.
Chander S. K., Lansdown A. G. B., Luqmani Y. A., Gomm J. J., Coope R. C., Gould M. N. & Coope R. C. Gould M. N. & Coombes R. C. Effectiveness of combined limonene and 4-hydroxyandrostenedione in the treatment of NMU-induced rat mammary tumors. Br. J. Cancer., 69:879-882.
Colombini M. P. et al. Characterization of the balm of an Egyptian mummy from the seventh century B. C. Studies in Conservation., 45:19-29,2000.
Conner D. E., Beuchat L. R. Sensitivity of heat stressed yeasts to essential oils of plants. Appl. Environ. Microbiol., 47:229-233,1984.
Cragg G. M. et al. Natural products in drug discovery and development. J Nat. Prod., 60:52-60,1997.
Cragg G. M., Newman D. J. Discovery and development of antineoplastic agents from natural sources. Cancer Invest., 17:153-163,1999.
Croteau R. Biosynthesis and catabolism of monoterpenoids. Chem. Rev., 87:929-954,1987.
Crowell P. L., Kennan W. S., Haag J. D., Ahmad S., Vedejs E. & Gould M. N. Chemoprevention of mammary carcinogenesis by hydroxylated derivatives of d-limonene. Carcinogenesis., 13:1261-1264,1992.
Crowell P. L., Elson, Bailey, H. H., C. E., Elegbede A., Haag J. H., Gould M. N. Human metabolism of the experimental cancer therapeutic agent d-limonene. Cancer Chemother. Pharmacol., 35:31-37,1994 (a).
Crowell P. L., Gould M. N. Chemoprevention and therapy of cancer by d-limonene. CRC. Crit. Rev. Oncogenesis., 5:1-22,1994 (b).
Crowell P. L. et al. Dietary Phytochemicals in Cancer Prevention and Treatment. Plenum Press, New York, 1996,
Crowell P. L. Prevention and therapy of cancer by dietary monoterpenes. J. Nutr., 129(3): 775S-778S,1999.
De Pooter H. L. et al. Essential oil of the leaves of three Pistacia species grown in Egypt. Flav. Fragr. J, 6:229-232,1991.
Dietrich D. R. & Swenberg J. A. The presence of α₂ᵤ-globulin is necessary for d-limonene promotion of male rat kidney tumors. Cancer Res., 51:3512-3517,1991.
Elegbede J. A., Elson C. E., Qureshi A., Tanner M. A. & Gould M. N. Inhibition of DMBA-induced mammary cancer by the monoterpene d-limonene. Carcinogenesis., 5:661-665,1984.
Elegbede J. A., Maltzman T. H., Verma A. K., Tanner M. A. & Gould M. N. Mouse skin tumor promoting activity of orange peel oil and d-limonene: a reevaluation. Carcinogenesis., 7:2047-2049,1986 (a).
Elegbede J. A., Elson C. E., Tanner M. A., Qureshi A. &Gould M. N. Regression of rat primary mammary tumors following dietary d-limonene. J. Natl. Cancer Inst., 76:323-325,1986 (b).
Elson C. E., Maltzman T. H., Boston J. L., Tanner M. A. & Gould M. N. Anti-carcinogenic activity of d-limonene during the initiation and promotion-progression stages of DMBA-induced rat mammary carcinogenesis. Carcinogenesis., 9:331-332,1988.
Elson C. E., Yu S. G. The chemoprevention of cancer by mevalonate-derived constituents of fruits and vegetables. J. Nutr., 124:607-614,1994.
Elson C. E. Suppression of mevalonate pathway activities by dietary isoprenoids: protective roles in cancer roles in cancer and cardiovascuar disease. J. Nutr., 125:1666S-1672S, 1995.
Evans E., Arneson D., Kovatch R., Supko J., Morton T., Siemann L., Cannon R., Tomaszewski J., Smith A. Toxicology and pharmacology of perillyl alcohol (NSC-641066) in rats and dogs. Proc. Am. Assoc. Cancer Res., 36:366,1995.
Farnsworth N. R. et al. Medicinal plants in therapy. Bull. World Health Organ., 63:965-981,1985.
Farnsworth N. R. The role of ethnopharmacology in drug development. Ciba Found Symp., 154:2-11,1990.
Giner-Larza Eva m. et al. On the anti-inflammatory and anti-phospholipase A₂ activity of extract from lanostane-rich species. ETH. J, 73:61-69,2000.
Gould M. N., Moore C. J., Zhang R., Wang B., Kennan W. S. & Haag J. D. Limonene chemoprevention of mammary carcinoma induction following direct in situ transfer of v-Ha-ras. Cancer Res., 54:3540-3543,1994.
Haag J. D., Lindstrom M. J. & Gould M. N. Limonene-induced regression of mammary carcinomas. Cancer Res., 52:4021-4026,1992.
Haag J. D. & Gould M. N. Mammary carcinoma regression induced by perillyl alcohol, a hydroxylated analog of limonene. Cancer. Chemother. Pharmacol., 34;477-483,1994.
Hursting S. D., Perkins S. N., Haines D. C., Ward J. M.& Phang J. M. Chemoprevention of spontaneous tumorigenesis in p53-knockout mice. Cancer Res., 55:3949-3953, 1995.
Huwez F. U. et al. Mastic Gum Kills Helicobacter pylori. N. Engl. J. Med., 339(26):365,1998.
Jeffers L., Church D., Gould M. and Wilding G. The effect of perillyl alcohol on the proliferation of human prostatic cell lines. Proc. Am. Assoc. Cancer Res., 36:303, 1995.
Jirtle R. L., Haag J: D., Ariazi E., Gould M. N. Increased mannose 6-phosphate/insulin-like growth factor II receptor and transforming growth fact b 1 levels during monoterpene-induced regression of mammary tumors. Cancer Res., 53:3849-3853, 1993.
Kawamori T., Tanaka T., Hirose Y., Ohnishi M. & Mori H. Inhibitory effects of d-limonene on the development of colonic aberrant crypt foci induced by azoxymethane in F344 rats. Carcinogenesis., 17:369-372,1996.
Kawata S., Nagase T., Yamasaki E., Ishiguro H., Matsuzwawa Y. Modulation of the mevalonate pathway and cell growth by pravastatin and d-limonene in a human hepatoma cell line (Hep G2). Br. J. Cancer., 69:1015-1020,1994.
Magiatis P. et al. Chemical Composition and Antimicrobial Activity of the Essential Oils of Pistacia lentiscus var. chia. Planta Med., 65(8):749-752,1999.
Maltzman T. H., Hurt L. M., Elson C. E., Tanner M. A. & Gould M. N. The prevention of nitrosomethylurea-induced mammary tumors by d-limonene and orange oil. Carcinogenesis., 10:781-785,1989.
Marone P. et al. Bacterial activity of Pistacia lentiscus mastic gum against Helicobacter pylori. J Chemoth., 13(6):611-614,2001.
McNamee D. Limonene trial in cancer. Lancet., 342:801,1993.
Mills J. J., Chari R. S., Boyer I. J., Gould M. N. & Jirtle R. L. Induction of apoptosis in liver tumors by the monoterpene perillyl alcohol. Cancer Res., 55:979-983,1995.
Papageorgiou V. P. et al. GLC-MS computer analysis of the essential oil of mastic gum. Chim. Chronica, New Ser., 10: 119-124,1981.
Phillips L. R., Malspeis L. & Supko J. G. Pharmacokinetics of active drug metabolites after oral administration of perillyl alcohol, an investigational antineoplastic agent, to the dog. Drug Metab. Dispos., 23:676-680.
Ray M., Kratz D., Lewis K., Hohl R. J. Effects of combinations of lovastatin and monoterpenes on ras processing. Proc of AACR., 36:428,1995.
Ren Z. and Gould M. N. Inhibition of ubiquinone and cholesterol synthesis by the monoterpene perillyl alcohol. Cancer Lett. 76:185-190, 1994.
Russin W. A., Hoesly J. D., Elson C. E., Tanner M. A. & Gould M. N. Inhibition of rat mammary carcinogenesis by monoterpenoids. Carcinogenesis., 10:2161-2165,1989.
Sanz M. J. et al. In vivo hypotensive activity of Pistacia lentiscus L. Phytother. Res., 2(4):201-203,1988.
Scurbis B., Markakis P. Essential oil of mastic gum. Int. Flavours Food Addit., 6:349,1975.
Shoff S. M., Grummer M., Yatvin M. B. & Elson C. E. Concentration-dependent increase of murine P388 and B 16 population doubling time by the acyclic monoterpene geraniol. Cancer Res., 51:37-42,1991.
Stark M. J. et al. Chemotherapy of pancreatic cancer with the monoterpene perillyl alcohol. Cancer lett., 96(1): 15-21,1995.
Wattenberg L. W. Inhibition of neoplasia by minor dietary constituents. Cancer Res., 43:2448s-2453s,1983.
Wattenberg L. W., Sparnins V. L. & Barany G. Inhibition of N-nitrosodiethylamine carcinogenesis in mice by naturally occurring organosulfur compounds and monoterpenes. Cancer Res., 49:2689-2694,1989.
Wattenberg L. W., & Coccia J. B. Inhibition of 4-(methylnitrosoamino)-1-butanone carcinogenesis in mice by d-limonene and citrus fruit oils. Carcinogenesis., 12:115-117,1991.
Wyllie S. G. et al. Volatile components of the fruits of Pistacia lentiscus. J. Food Sci., 55:1325-1326,1990.
Yu S. G., Hildebrandt L. A. & Elson C. E. Geraniol, an inhibitors of mevalonate biosynthesis, suppresses the growth of hepatomas and melanomas transplanted to rats and mice. J. Nutr., 125:2763-2767,1995.

1. A method for treating or preventing cancer in a mammal, including a human, comprising administrating an effective amount of a product obtained from a plant of Pistacia genus.

2. The method of claim 1 wherein the product is selected from the group consisting of natural products of a Pistacia genus plant, essential oils from a Pistacia genus plant or parts thereof, natural or synthetic components of essential oils from a Pistacia genus plant or parts thereof and mixtures and derivatives thereof.

3. The method of claim 1 wherein the product is selected from the group consisting of natural products of a Pistacia genus plant containing essential oils, essential oils from a Pistacia genus plant or parts thereof, natural or synthetic components of essential oils from a Pistacia genus plant or parts thereof, mixtures and derivatives thereof.

4. The method of claim 1 wherein the product is an essential oil obtained from plants of pistacia genus or components thereof.

5. The method of claim 1 wherein the product is a product obtained from essential oils of plants of Pistacia genus or components thereof.

6. The method of claim 1, wherein the plants of Pistacia genus are of European origin.

7. The method of claim 1, wherein the plants of the Pistacia genus are selected from the group consisting of p. Terebinthus, p. Lentiscus and p. Vera.

8. The method of claim 7 wherein the essential oils are essential oils obtained from twigs of p. Lentiscus of Portuguese origin.

9. The method of claim 7 wherein the essential oils are essential oils obtained from leaves or twigs of p. Lentiscus of Italian origin.

10. The method of claim 9 wherein the essential oils are essential oils obtained from leaves or twigs of p. Lentiscus of Tuscany.

11. The method of claim 7 wherein the essential oils are selected from the group consisting of essential oils obtained from galls of p. Terebinthus of Italian origin, essential oils obtained from leaves or twigs of p. Terebinthus of Italian origin and essential oils obtained from fruits of p. Terebinthus of Italian origin.

12. The method of claim 11 wherein the essential oils are essential oils obtained from galls of p. Terebinthus of Veneto region.

13. The method of claim 7 wherein the essential oils are essential oils obtained from p. Vera of Italian origin.

14. The method of claim 13 wherein the essential oils are essential oils obtained from seeds of p. Vera of Sicily.

15. The method of claim 1 wherein the cancer is a cancer selected from the group consisting of breast adenocarcinoma, colon adenocarcinoma and ovary adenocarcinoma.

16. The method of claim 15 wherein the cancer is breast adenocarcinoma and the essential oils are selected from the group consisting of essential oils obtained from p. Lentiscus from Portugal, essential oils obtained from p. Lentiscus from Italy, essential oils obtained from p. Terebinthus of Italian origin and essential oils obtained from p. Vera from Italy.

17. The method of claim 16 wherein the cancer is breast adenocarcinoma and the essential oils are selected from the group consisting of essential oils obtained from twigs p. Lentiscus from Portugal, essential oils obtained from leaves of p. Lentiscus from Italy, essential oils obtained from galls of p. Terebinthus of Italian origin, essential oils obtained from twigs of p. Terebinthus of Italian origin and essential oils obtained from seeds of p. Vera from Italy.

18. The method of claim 17 wherein the cancer is ovary adenocarcinoma and the essential oils are essential oils obtained from p. Lentiscus from Portugal, essential oils essential oils from p. Lentiscus from Italy and essential oils obtained from p. Terebinthus of Italian origin.

19. The method of claim 15 wherein the cancer is ovary adenocarcinoma and the essential oils are essential oils obtained from leaves of p. Lentiscus from Portugal, essential oils essential oils from leaves of p. Lentiscus from Italy, essential oils obtained from twigs of p. Terebinthus of Italian origin, essential oils obtained from fruits of p. Terebinthus of Italian origin and essential oils obtained from galls of p. Terebinthus of Italian origin.

20. The method of claim 15 wherein the cancer is colon adenocarcinoma and the essential oils are obtained from leaves of p. Lentiscus from Portugal.

21. The method of claim 4 wherein the essential oil component is (−)-bornyl acetate and the cancer is selected from the group consisting of colon adenocarcinoma and ovary adenocarcinoma.
